(19) United States
(12) Patent Application Publication
(10) Pub. No.: US 2025/0000487 A1
(43) Pub. Date: Jan. 2, 2025

(54) ULTRASONIC PROBE AND ULTRASONIC DIAGNOSTIC SYSTEM

(71) Applicant: Socionext Inc., Kanagawa (JP)

(72) Inventor: Naoto ADACHI, Yokohama (JP)

(21) Appl. No.: 18/882,246

(22) Filed: Sep. 11, 2024

Related U.S. Application Data
(63) Continuation of application No. PCT/JP2022/012446, filed on Mar. 17, 2022.

(51) Int. Cl. A61B 8/00

(52) U.S. Cl. CPC A61B 8/4444; A61B 8/4483; A61B 8/461

(57) ABSTRACT

An ultrasonic probe includes a transducer, a self-diagnosis circuit configured to perform inspection of the transducer, and an output part configured to output information that is in accordance with a result of the inspection performed by the self-diagnosis circuit.

### CROSS-REFERENCE TO RELATED APPLICATIONS

This application is a continuation application of International Application No. PCT/JP2022/012446 filed on Mar. 17, 2022, and designated the U.S., the entire contents of which are incorporated herein by reference.

### BACKGROUND

### 1. Field of the Invention

The present disclosure relates to an ultrasonic probe and an ultrasonic diagnostic system.

### 2. Description of the Related Art

An ultrasonic diagnostic system transmitting ultrasonic waves from the surface of a body to the interior thereof, receiving reflected waves, and imaging the received reflected waves using an ultrasonic probe to diagnose the presence or absence, size, shape, depth, or the like of a lesion, is known.

In the ultrasonic diagnostic system, a diagnostic method for diagnosing the condition of an ultrasonic transducer (hereinafter referred to as “transducer”) that transmits and receives ultrasonic waves, is known. See, for example, Japanese Laid-Open Patent Application Nos. 1985-193447 and 1996-238243, and International Publication No. WO2008/035415.

### SUMMARY

An ultrasonic probe according to an embodiment of the present disclosure includes a transducer, a self-diagnosis circuit configured to perform inspection of the transducer, and an output part (output circuit) configured to output information that is in accordance with a result of the inspection performed by the self-diagnosis circuit.

### BRIEF DESCRIPTION OF THE DRAWINGS

FIG. 1 is a diagram illustrating a configuration example of an ultrasonic probe according to a first embodiment;

FIG. 2 is a diagram illustrating a configuration example of a self-diagnosis circuit according to the first embodiment;

FIG. 3 is a flowchart illustrating an example of self-diagnosis processing according to the first embodiment;

FIG. 4 is a diagram illustrating a transducer according to the first embodiment;

FIG. 5 is a flowchart illustrating another example of the self-diagnosis processing according to the first embodiment;

FIG. 6 is a flowchart illustrating an example of self-diagnosis processing according to a second embodiment;

FIG. 7 is a diagram illustrating a configuration example of an ultrasonic diagnostic system according to the second embodiment;

FIG. 8 is a sequence diagram illustrating an example of inspection result display processing according to the second embodiment;

FIG. 9 is a diagram illustrating an example of a terminal display screen according to the second embodiment;

FIG. 10 is a diagram (1) illustrating a configuration example of an ultrasonic diagnostic system; and

FIG. 11 is a diagram (2) illustrating a configuration example of the ultrasonic diagnostic system.

### DETAILED DESCRIPTION OF THE INVENTION

Existing techniques use a display of an ultrasonic diagnostic system to determine the condition of a transducer of an ultrasonic probe, and there is an issue that the condition of the transducer cannot be diagnosed by the ultrasonic probe alone.

An embodiment of the present disclosure is made in view of the above. The present disclosure provides an ultrasonic diagnostic system configured to diagnose the condition of the transducer by the ultrasonic probe itself, without using the display of the ultrasonic diagnostic system.

Embodiments of the present disclosure will be described below with reference to the accompanying drawings.

### <Ultrasonic Diagnostic System>

Before describing the ultrasonic probe and the ultrasonic diagnostic system according to the present embodiment, an outline of an ultrasonic diagnostic system as a premise of the present embodiment will be described.

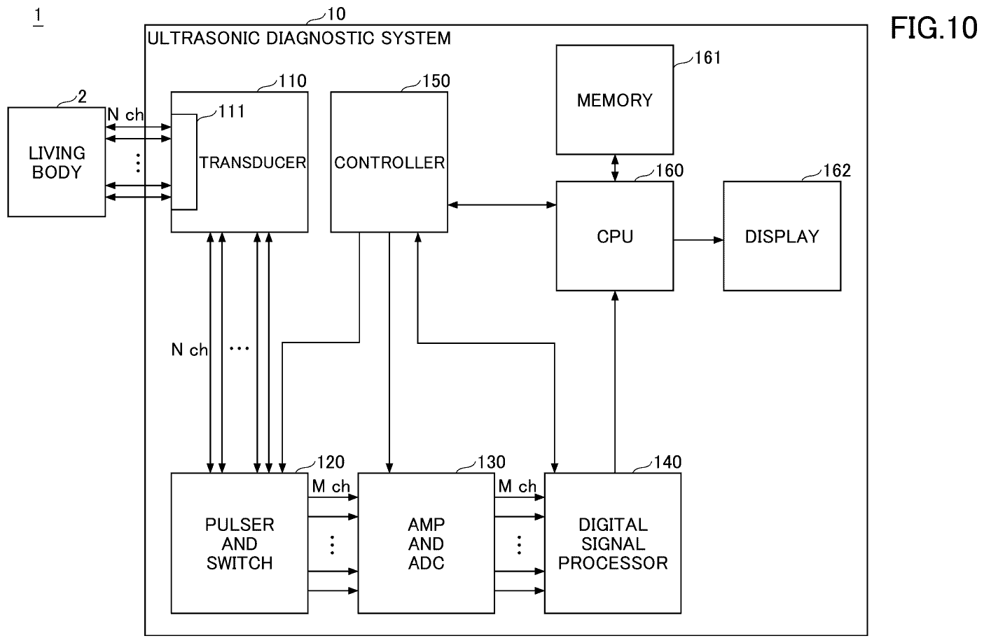

FIG. 10 is a diagram (1) illustrating a configuration example of an ultrasonic diagnostic system. An ultrasonic diagnostic system 1 is a system configured to transmit ultrasonic waves from the surface of a body to the interior thereof, receive reflected waves, and image the received reflected waves using an ultrasonic transducer (hereinafter referred to as “transducer”) to diagnose the presence or absence, size, shape, depth, or the like of a lesion. The transducer may also be called, for example, a probe or the like.

In the example as illustrated in diagram (1), the ultrasonic diagnostic system 1 includes an ultrasonic diagnostic device 10. The ultrasonic diagnostic device 10 includes, for example, a transducer 110, a pulser and switch 120, an amplifier (AMP) and analog-to-digital converter (ADC) 130, a digital signal processor 140, a controller 150, a central processing unit (CPU) 160, a memory 161, a display 162, and the like.

The transducer 110 includes an N-channel (for example, 128 channels) oscillator array 111 in which a plurality of oscillators are arranged in an array. The transducer 110 uses an oscillator selected by the pulser and switch 120, and transmits ultrasonic waves and receives ultrasonic waves reflected by a living body 2 or the like.

The switch of the pulser and switch 120 selects an oscillator of the oscillator array 111. The pulser of the pulser and switch 120 transmits a pulse signal to the transducer 110, and irradiates the living body 2 and the like with ultrasonic waves from the transducer 110.

In response to irradiation with the ultrasonic wave, the living body 2 reflects the ultrasonic wave at a boundary where the acoustic impedance is different. The wave reflected from the living body 2 is received by the transducer 110, and the received signal is output to the AMP and ADC 130 selected by the switch of the pulser and switch 120. At this time, the pulser and switch 120 outputs the M channel number of received signals (for example, 64 channels) to the AMP and ADC 130. The N number of oscillators (128 channels) included in the transducer 110 and the number M of received signals (64 channels) output by the pulser and switch 120 are examples, and may be any other number.

The AMP and ADC 130 is configured to: amplify, with the AMP, the received signal of the ultrasonic waves output from the pulser and switch 120; convert the amplified signal into a digital signal with the ADC; and output the converted signal to the digital signal processor 140.

The digital signal processor 140 is configured to perform various digital signal processes on the digital signal output from the AMP and ADC 130. For example, the digital signal processor 140 performs: delay adjustment processing that adjusts delay in signal reception timing for the M number of channels input; averaging (delay-and-sum) processing; gain correction processing considering attenuation in the living body 2; envelope processing that extracts luminance information; and the like. In addition, the digital signal processor 140 performs the above processing, and outputs obtained image data to the CPU 160 via an interface, such as a serial peripheral interface (SPI) or the like.

The CPU 160 is a processor configured to perform various programmed controls by executing predetermined programs. The CPU 160 displays image data received from the digital signal processor 140, on a display 162 or the like.

The controller 150 is configured to output a control signal controlling the pulser and switch 120, the AMP and ADC 130, the digital signal processor 140, and the like in accordance with an instruction from the CPU 160, for example.

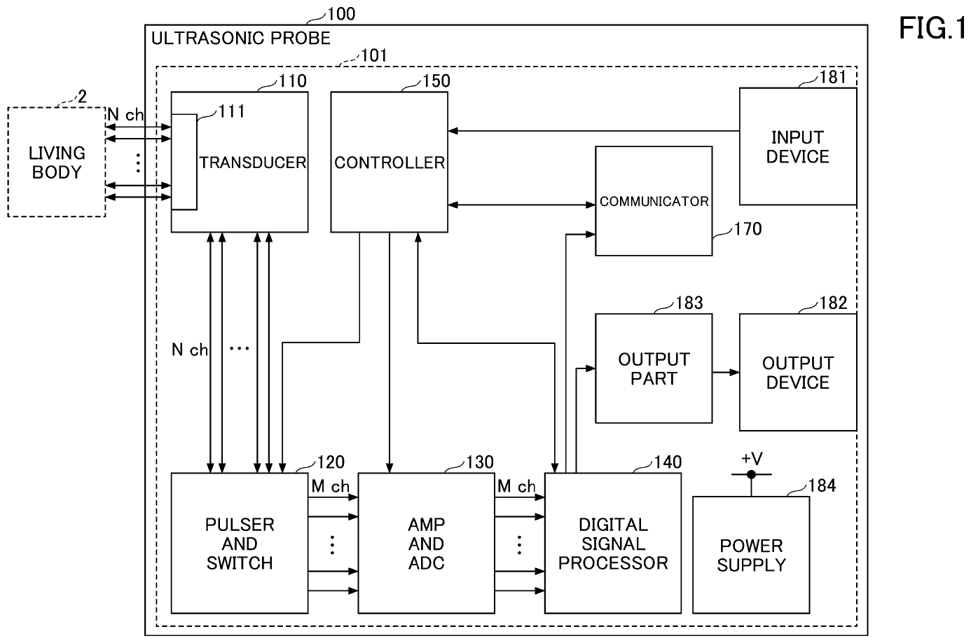

With the configuration of FIG. 1, the ultrasonic diagnostic system 1 can display, on the display 162, an ultrasonic image for diagnosing the presence or absence, size, shape, depth, or the like of a lesion in the interior of the living body 2.

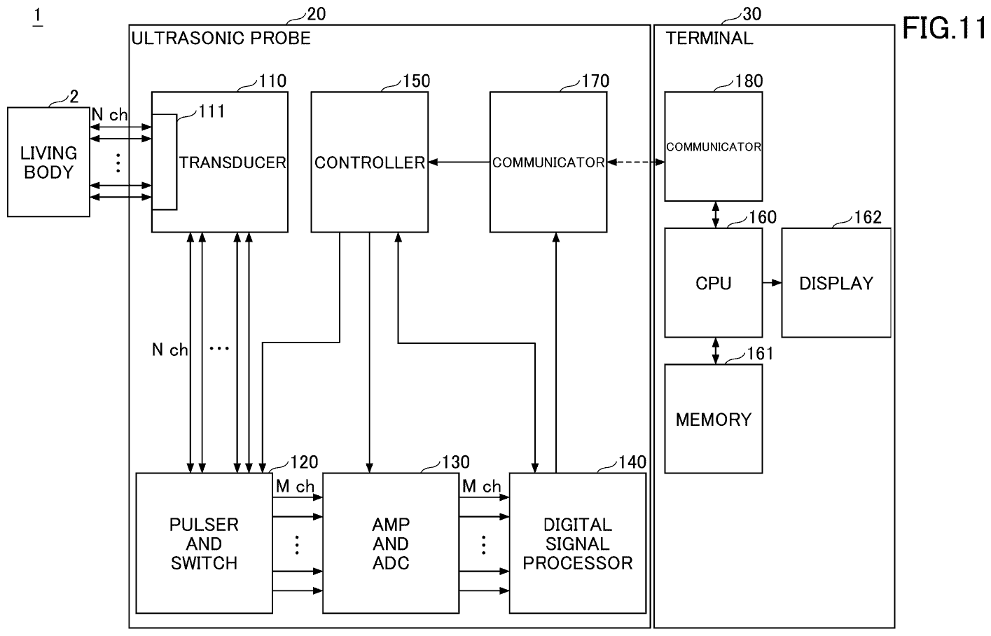

As another example, the ultrasonic diagnostic system 1 may include an ultrasonic probe 20 and a terminal 30, as illustrated in FIG. 11.

FIG. 11 is a diagram (2) illustrating a configuration example of the ultrasonic diagnostic system 1. In the example of FIG. 11, the ultrasonic diagnostic system 1 includes an ultrasonic probe 20 and a terminal 30.

The ultrasonic probe 20 includes the transducer 110, the pulser and switch 120, the AMP and ADC 130, the digital signal processor 140, and the controller 150, which are as described in FIG. 1, and a communicator 170 configured to wirelessly communicate with the terminal 30.

The communicator 170 transmits and receives data to and from the terminal 30 in various types of wireless communications, for example, a wireless local area network (LAN), near-field wireless communication, or ultra-wideband (UWB). The communicator 170 is not limited to use of the wireless communication, but may also communicate with the terminal 30 through wired communication.

The terminal 30 is, for example, a general-purpose information terminal, such as a personal computer (PC), a smartphone, a tablet terminal, or the like. The terminal 30 is configured to receive ultrasonic image data from the ultrasonic probe 20 by wireless communication by executing an application program corresponding to the ultrasonic diagnostic system 1, for example, and display an ultrasonic image on the display 162. Further, the terminal 30 displays an operation screen for starting, finishing, setting, or the like of the diagnosis of the ultrasonic diagnostic system 1, and transmits a control signal to the ultrasonic probe 20 in response to the operation on the operation screen.

The terminal 30 is not limited to a general-purpose information terminal, and may be a dedicated terminal incorporating firmware corresponding to the ultrasonic diagnostic system 1.

In the ultrasonic diagnostic system 1, when each of the channels (N channels) of the transducer 110 is not properly connected, the image quality deteriorates. Since the deterioration in the image quality leads to misdiagnosis, it matters that each of the channels of the transducer 110 is properly connected in the ultrasonic diagnostic system 1.

In the ultrasonic diagnostic system, a diagnostic method for diagnosing the condition of the transducer transmitting and receiving ultrasonic waves is known. See, for example, Japanese Laid-Open Patent Application Nos. 1985-193447 and 1996-238243, and International Publication No. WO2008/035415.

However, existing techniques use the display of the ultrasonic diagnostic system for determining the condition of the transducer of the ultrasonic probe, and there is an issue that the condition of the transducer cannot be diagnosed by the ultrasonic probe alone.

In the present embodiment, the condition of the transducer can be diagnosed by the ultrasonic probe alone without using the display of the ultrasonic diagnostic system. For this purpose, an ultrasonic probe 100 according to the present embodiment has, for example, the configuration as illustrated in FIG. 1.

### First Embodiment

### <Configuration of Ultrasonic Probe>

FIG. 1 is a diagram illustrating a configuration example of an ultrasonic probe according to the first embodiment. The ultrasonic probe 100 according to the first embodiment includes a transducer 110, a pulser and switch 120, an AMP and ADC 130, a digital signal processor 140, a controller 150, a communicator 170, an input device 181, an output device 182, an output part 183, a power supply 184, and the like.

The transducer 110 includes an N-channel oscillator array 111 in which a plurality of oscillators are arranged in an array. The transducer 110 transmits ultrasonic waves and receives reflected ultrasonic waves by the oscillator selected by the pulser and switch 120. In the present embodiment, a living body 2 is unnecessary when executing the self-diagnosis processing described below.

The switch of the pulser and switch 120 selects the oscillator from the oscillator array 111 by the switch, transmits a pulse signal to the transducer 110, and causes the transducer 110 to transmit an ultrasonic wave. The pulser and switch 120 outputs the received signal of the ultrasonic wave received by the transducer 110 to the AMP and ADC 130.

The AMP and ADC 130 amplifies, with the AMP, the received signal of the ultrasonic wave output from the pulser and switch 120, converts the amplified signal into a digital signal with the ADC, and outputs the converted signal to the digital signal processor 140.

The digital signal processor 140 performs various types of digital signal processing on the digital signal output from the AMP and ADC 130.

The controller 150 outputs a control signal for controlling the pulser and switch 120, the AMP and ADC 130, the digital signal processor 140, and the like, and controls the entire ultrasonic probe 100. When the controller 150 of the present embodiment receives a start operation of the self-diagnosis processing by the input device 181, the controller 150 controls the self-diagnosis processing, which will be described below.

The controller 150 may be implemented by hardware or by a computer, such as a microcomputer or the like, and a program performed by the computer.

The communicator 170 transmits and receives data, control signals, and the like to and from the terminal 30 through various wireless communications such as, for example, a wireless LAN, various types of short-range wireless communications, or UWB. The communicator 170 may communicate with the terminal 30 through wired communications, and is not limited to wireless communications. In the present embodiment, the communicator 170 is not used when executing the self-diagnosis processing described later.

The input device 181 is an input device such as, for example, a switch, a button, or a microphone that receives an operation to start self-diagnosis processing of the ultrasonic probe 100. Hereinafter, as one example, the input device 181 is referred to as a switch that receives an operation to start self-diagnosis processing of the ultrasonic probe 100.

The output device 182 is, for example, a light-emitting device, such as a light emitting diode (LED) or a sounding device, such as a buzzer or a speaker.

The output part 183 is configured to output information based on the result of the inspection by the self-diagnosis circuit 210 described later using the output device 182. For example, when the self-diagnosis circuit 210 detects a failure, the output part 183 causes the output device 182, such as an LED, to emit light with a different emission color or a different emission method (with or without flashing, interval between flashes, or the like) than when the failure is not detected. The light emission by the output device 182 is an example of display information indicating whether or not one or more channels of the transducer 110 have a failure.

As another example, when the self-diagnosis circuit 210 detects a failure, the output part 183 may output from the output device 182, such as a buzzer or a speaker, an alarm sound or a voice message that is different from the case where the failure is not detected. Note that the alarm sound or voice message output by the output device 182 is an example of sound information indicating whether or not there is a failure in one or more channels of the transducer 110.

The power supply 184 is, for example, a secondary battery capable of charging and discharging, and supplies power to each part of the ultrasonic probe 100.

### (Self-Diagnosis Circuit)

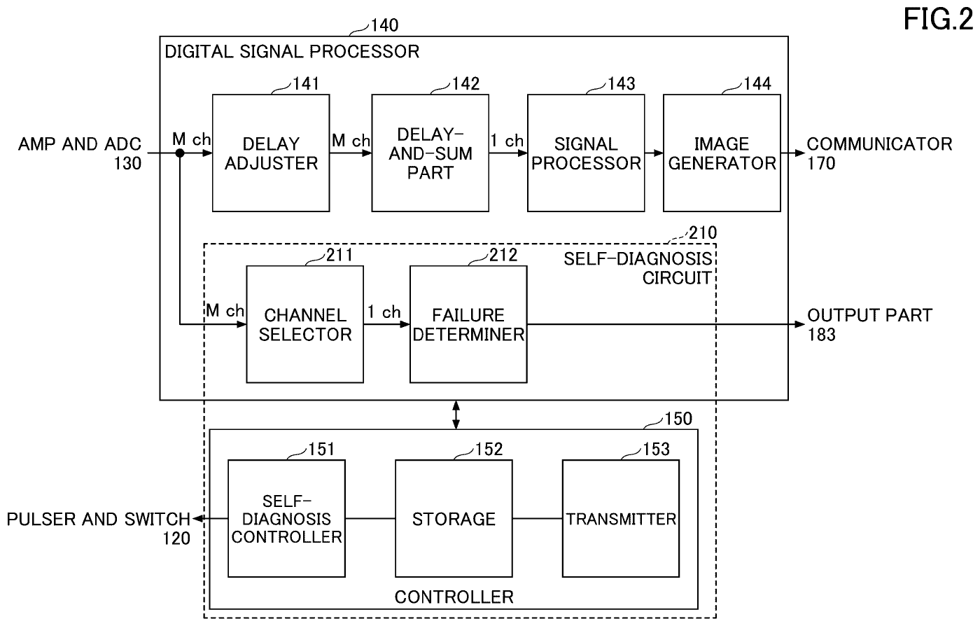

FIG. 2 is a diagram illustrating a configuration example of the self-diagnosis circuit according to the first embodiment. The digital signal processor 140 includes a delay adjuster 141 for executing the aforementioned delay adjustment processing, a delay-and-sum part 142 for executing averaging (delay-and-sum) processing, a signal processor 143 for executing gain correction processing, and an image generator 144 for executing envelope processing, or the like. The components of the delay adjuster 141, the delay-and-sum part 142, the signal processor 143, and the image generator 144 are used in the normal operation mode for executing ultrasonic diagnosis of the living body 2.

In addition to the above components, the digital signal processor 140 according to the present embodiment includes a channel selector 211 and a failure determiner 212. Further, the controller 150 includes a self-diagnosis controller 151, a storage 152, and a transmitter 153. The storage 152 and/or the transmitter 153 may be provided externally of the controller 150.

The self-diagnosis controller 151 is configured to control the self-diagnosis processing that determines the presence or absence of a failure in each channel of the transducer 110. The storage 152 is configured to store inspection results and the like of the self-diagnosis processing. The transmitter 153 is configured to transmit inspection results stored in the storage 152 to the exterior, for example, in response to a request from the terminal 30.

The ultrasonic probe 100 is provided with a self-diagnosis circuit 210 that inspects the transducer 110 by the channel selector 211, the failure determiner 212, the self-diagnosis controller 151, the storage 152, the transmitter 153, and the like. The self-diagnosis circuit 210 is used in a self-diagnosis mode that performs the self-diagnosis processing.

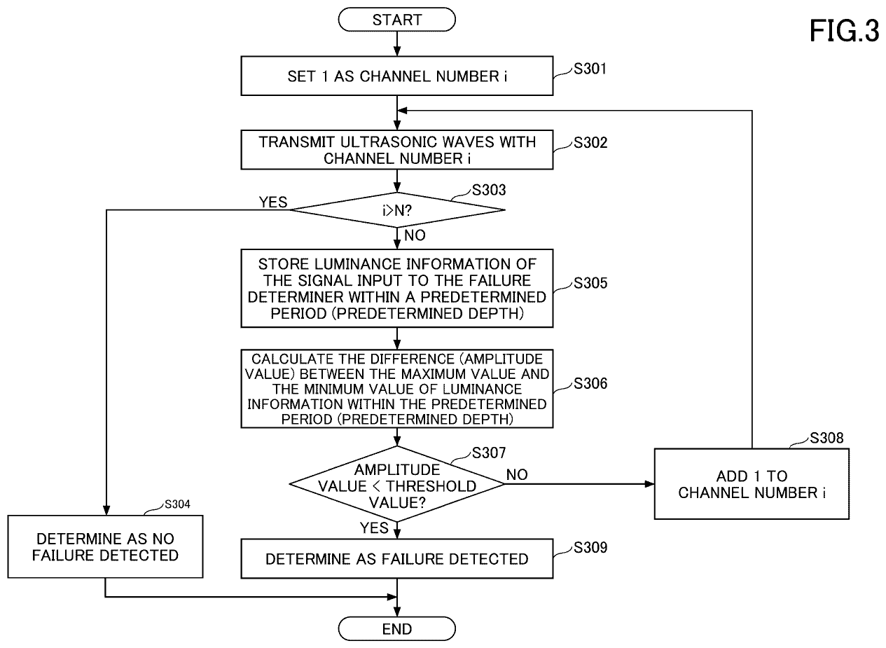

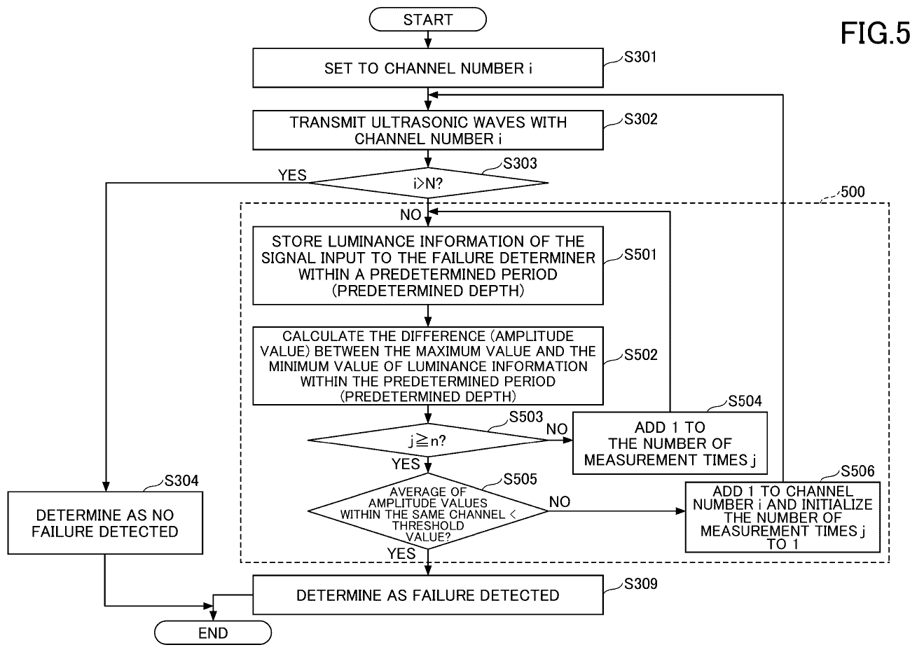

The self-diagnosis circuit 210 performs self-diagnosis processing as illustrated in FIGS. 3 and 5, for example, in the self-diagnosis mode.

Preferably, the ultrasonic probe 100 includes a single housing 101 that includes the components as described in FIGS. 1 and 2.

### <Flow of Processing>

### (Self-Diagnosis Processing 1)

FIG. 3 is a flowchart illustrating an example of the self-diagnosis processing according to the first embodiment. This processing shows an example of the self-diagnosis processing performed when the ultrasonic probe 100 as described in FIGS. 1 and 2 receives an operation to start the self-diagnosis processing from the input device 181. The processing as illustrated in FIG. 3 is performed when the transducer 110 is not contacted with the living body 2 or the like.

In step S301, the self-diagnosis circuit 210 sets “1” as the channel number i.

In step S302, the self-diagnosis circuit 210 transmits ultrasonic waves with the channel number 1 of the transducer 110.

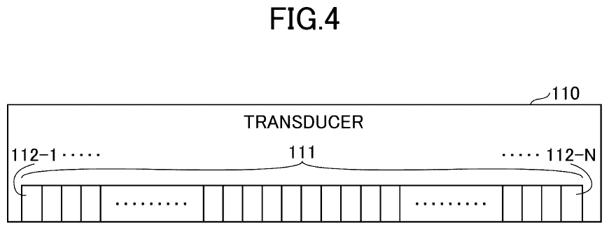

FIG. 4 is a diagram illustrating the transducer according to the first embodiment. The transducer 110 according to the first embodiment includes an oscillator array 111. The oscillator array 111 includes N oscillators 112-1 to 112-N arranged in a row.

In step S302 of FIG. 3, the self-diagnosis controller 151 controls, for example, the pulser and switch 120 to transmit ultrasonic waves from an oscillator 112-i among the N oscillators 112-1 to 112-N of the transducer 110.

In step S303, the self-diagnosis circuit 210 determines whether or not the processes in steps S305 to S309 have been performed for the channel numbers 1 to N by determining whether or not i>N. When i>N, the self-diagnosis controller 151 proceeds the processing to step S304. On the other hand, when i>N is not satisfied, the self-diagnosis circuit 210 proceeds the processing to step S305.

When the processing proceeds to step S304, the self-diagnosis circuit 210 determines that there is no failure detection (there is no failure in each channel of the transducer 110), and outputs a signal indicating that there is no failure to the output part 183.

In response, the output part 183 uses the output device 182 to output information indicating that there is no failure in the transducer 110. For example, the output part 183 may cause a light-emitting device such as an LED, which is an example of the output device 182, to emit light in a color (e.g., green) indicating that there is no failure, or may display the light-emitting device lit in any color. As another example, the output part 183 may output a passing sound or a voice message indicating that there is no failure from a sounding element such as a buzzer or a speaker, which is another example of the output device 182.

In step S305, the self-diagnosis circuit 210 stores luminance information of the signal input to the failure determiner 212 within a predetermined period (predetermined depth). For example, the self-diagnosis controller 151 causes the channel selector 211 to select a signal corresponding to the channel number i from the input signal of the M channels input from the AMP and ADC 130. The failure determiner 212 also obtains luminance information of the input signal for a predetermined period (predetermined depth) and stores it.

In step S306, the self-diagnosis circuit 210 calculates a difference (hereinafter referred to as “amplitude value”) between the maximum value and the minimum value of luminance information within the predetermined period (predetermined depth).

In step S307, the self-diagnosis circuit 210 determines whether or not the calculated amplitude value is smaller than a predetermined threshold value (failure threshold value). If the amplitude value is smaller than the threshold value, the self-diagnosis circuit 210 proceeds the processing to step S309. On the other hand, if the amplitude value is not smaller than the threshold value, the self-diagnosis circuit 210 proceeds the processing to step S308.

When the processing proceeds to step S308, the self-diagnosis circuit 210 adds 1 to the channel number i and returns the processing to step S302.

When the processing proceeds to step S309, the self-diagnosis circuit 210 determines that a failure has been detected (a failure exists in one of the channels of the transducer 110) and outputs a signal indicating that a failure exists to the output part 183.

In response, the output part 183 uses the output device 182 to output information indicating that a failure exists in the transducer 110. For example, the output part 183 may cause a light-emitting device such as an LED, which is an example of the output device 182, to emit light in a color (e.g., red) indicating that a failure exists, or may display the light-emitting device with flashing in any color. As another example, the output part 183 may output an alarm sound, a voice message, or the like indicating that a failure exists from a sounding element such as a buzzer or a speaker, which is another example of the output device 182.

By the processing in steps S302 to S309, the self-diagnosis circuit 210 causes the plurality of channels of the transducer 110 to transmit and receive signals one by one in order, and determines that the channel whose difference between the maximum value and the minimum value of the received signals within a predetermined period is smaller than the threshold value, is with a failure.

When the transducer 110 is not contacted with the living body 2 or the like, a constant amplitude value is obtained at the depth of the surface layer by multiple reflection with air when the signal is correctly connected. On the other hand, when the signal is not correctly connected, the amplitude value becomes smaller than when the signal is correctly connected.

Therefore, when the amplitude value within the range (depending on the frequency of the transducer used, for example, approximately a depth of 5 mm to 10 mm), when the depth at which the multiple reflections are observed is the display depth, is smaller than a predetermined threshold value (failure threshold value), it can be determined that there is a failure. Conversely, when the amplitude value exceeds the threshold value in all channels after sweeping all channels of the transducer 110, it can be determined that there is no failure.

### (Self-Diagnosis Processing 2)

FIG. 5 is a flowchart illustrating another example of the self-diagnosis processing according to the first embodiment. This processing shows another example of the self-diagnosis processing performed when the ultrasonic probe 100 as described in FIGS. 1 and 2 receives an operation to start self-diagnosis processing from the input device 181. Among the processes as illustrated in FIG. 5, the processes in steps S301 to S304 and S309 are the same as those described in FIG. 3, and therefore, the description thereof is omitted here. Further, a detailed description of the processing contents similar to the processing as described in FIG. 3 will be omitted here.

When the processing proceeds from step S303 to step S501, the self-diagnosis circuit 210 stores luminance information of the signal input to the failure determiner 212 within a predetermined period (predetermined depth).

In step S502, the self-diagnosis circuit 210 calculates a difference (amplitude value) between the maximum value and the minimum value of luminance information within the predetermined period (predetermined depth).

In step S503, the self-diagnosis circuit 210 determines whether or not the value of the number of measurement times j within the same channel is equal to or greater than the predetermined number of measurement times n (whether or not the predetermined number of measurement times n has been reached). When the value of j is not equal to or greater than n, the self-diagnosis circuit 210 proceeds the processing to step S504. On the other hand, if the value of j is equal to or greater than n, the self-diagnosis circuit 210 proceeds the processing to step S505.

When the processing proceeds to step S504, the self-diagnosis circuit 210 adds 1 to j and returns the processing to step S501.

When the processing proceeds to step S505, the self-diagnosis circuit 210 determines whether or not the average of the amplitude values within the same channel is smaller than a predetermined threshold value (failure threshold value). When the average of the amplitude values within the same channel is smaller than the threshold value, the self-diagnosis circuit 210 proceeds the processing to step S309. On the other hand, when the average of the amplitude values within the same channel is not smaller than the threshold value, the self-diagnosis circuit 210 proceeds the processing to step S506.

When the processing proceeds to step S506, the self-diagnosis circuit 210 adds 1 to the channel number i, initializes the number of measurement times j to 1, and returns the processing to step S302.

By the processing as illustrated in FIG. 5, the self-diagnosis circuit 210 performs the processes in steps S501 and S502 multiple times within the same channel, thereby improving the determination accuracy of failure detection.

### Second Embodiment

In the first embodiment, when any channel of the transducer 110 has a failure, the self-diagnosis circuit 210 determines that a failure has been determined and terminates the processing. However, the self-diagnosis circuit 210 may perform failure determination for all channels of the transducer 110.

### (Self-Diagnosis Processing 3)

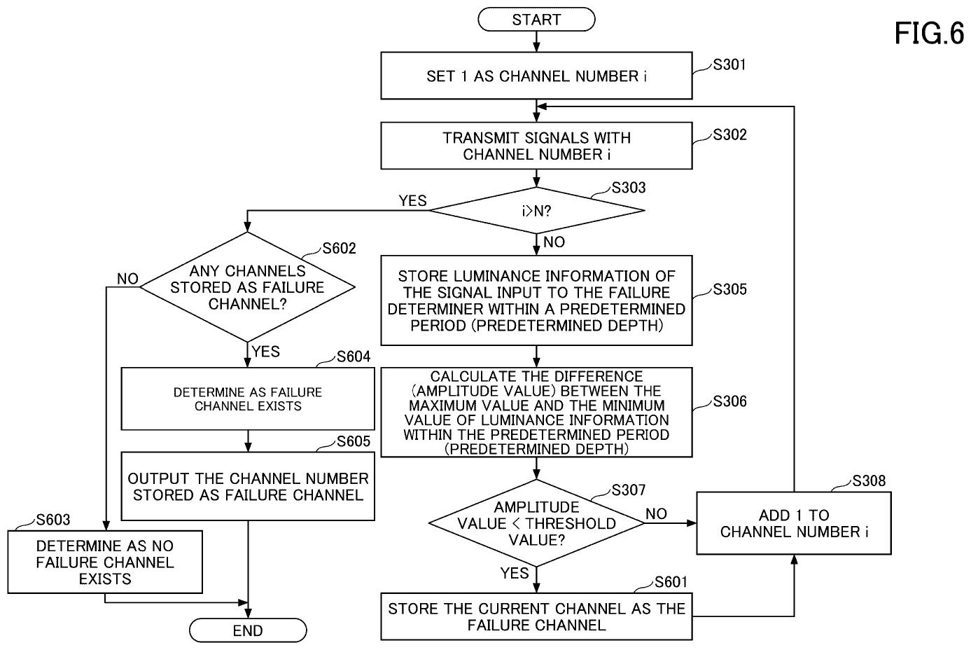

FIG. 6 shows an example of self-diagnosis processing according to the second embodiment. This processing shows another example of self-diagnosis processing performed when the ultrasonic probe 100 as described in FIGS. 1 and 2 receives an operation to start self-diagnosis processing from the input device 181. Among the processes as illustrated in FIG. 6, the processes in steps S301 to S303 and S305 to S308 are similar to the processes as described in FIG. 3, and therefore, differences from the processes as described in FIG. 3 will be described here.

When the amplitude value is smaller than the threshold value in step S307, the self-diagnosis circuit 210 proceeds the processing to step S601.

When the processing proceeds to step S601, the self-diagnosis circuit 210 stores the current channel as a failure channel in the storage 152, and proceeds the processing to step S308. By this processing, the self-diagnosis circuit 210 stores the information of the channel determined to have a failure in the storage 152.

When the processing proceeds from step S303 to step S602, the self-diagnosis circuit 210 determines whether or not the storage 152 has a channel that has been stored as a failure channel. When there is no channel that has been stored as a failure channel, the self-diagnosis circuit 210 proceeds the processing to step S603. On the other hand, when there is a channel that has been stored as a failure channel, the self-diagnosis circuit 210 proceeds the processing to step S603.

When the processing proceeds to step S603, the self-diagnosis circuit 210 determines that there is no failure detection (there is no failure in each channel of the transducer 110), and outputs a signal indicating that there is no failure to the output part 183.

On the other hand, when the processing proceeds to step S604, the self-diagnosis circuit 210 determines that there is failure detection (there is a failure in any channel of the transducer 110), and outputs a signal indicating that there is a failure to the output part 183.

In step S605, the self-diagnosis circuit 210 outputs the channel number stored as the failure channel. For example, the self-diagnosis circuit 210 may use the output part 183 to change the color of a light-emitting device such as an LED or the blinking speed. Alternatively, the self-diagnosis circuit 210 may output the channel number stored as the failure channel to the storage 152 and transmit the channel number stored as the failure channel to the terminal 30 or the like in response to a request from the terminal 30 or the like.

### (System Configuration)

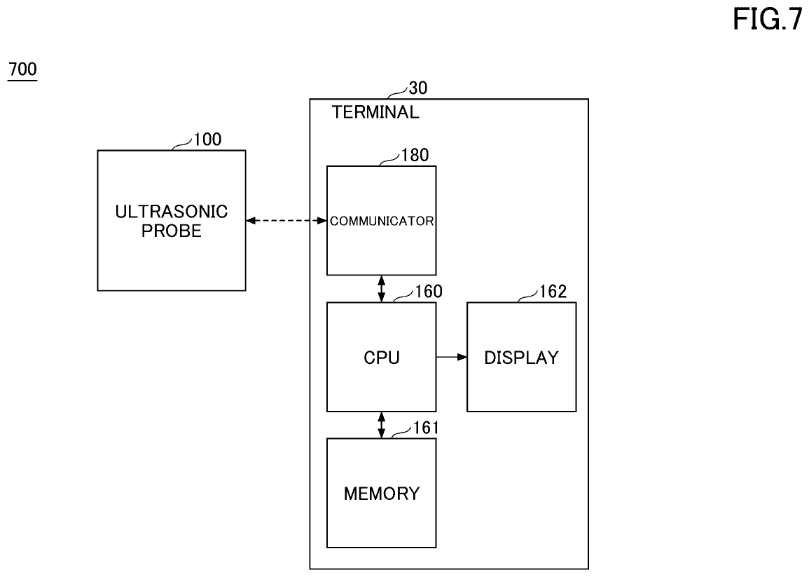

FIG. 7 is a diagram illustrating a configuration example of an ultrasonic diagnostic system according to the second embodiment. The ultrasonic diagnostic system 700 according to the second embodiment includes an ultrasonic probe 100 as described in FIGS. 1 and 2 and a terminal 30 capable of communicating with the ultrasonic probe 100 by wireless communications or wired communications.

The terminal 30 is a general-purpose information terminal such as, for example, a PC, a smartphone, or a tablet terminal, and can communicate with the ultrasonic probe 100 using the communicator 180 by executing a predetermined program stored in the memory 161 or the like by the CPU 160. Moreover, the terminal 30 can obtain the inspection result of the transducer 110 from the ultrasonic probe 100 and display the inspection result on the display 162 in response to the operation to display the inspection result by the user.

### (Processing to Display the Inspection Result)

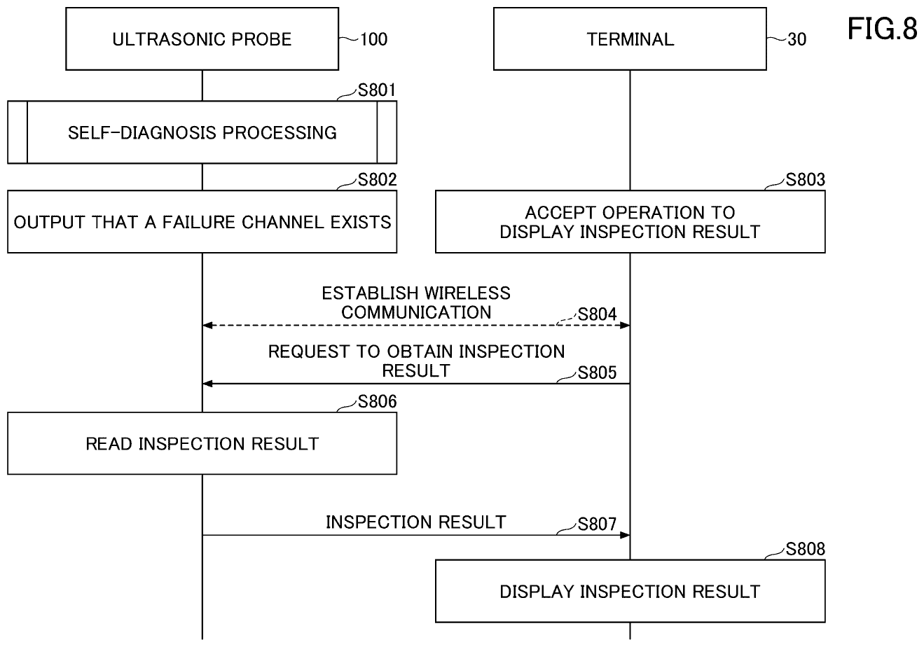

FIG. 8 is a flowchart illustrating an example of processing to display an inspection result according to the second embodiment. This processing shows an example of the processing performed by the ultrasonic diagnostic system 700 when the ultrasonic probe 100 performs the self-diagnosis processing as described in FIG. 6 and a failure channel is found in the transducer 110.

In step S801, the self-diagnosis circuit 210 of the ultrasonic probe 100 performs, for example, the self-diagnosis processing as described in FIG. 6. The processing as illustrated in FIG. 6 is an example of the self-diagnosis processing according to the second embodiment. For example, the self-diagnosis circuit 210 may perform the processing 500 in the steps S501 to S506 in FIG. 5 instead of the processing in the steps S305 to S308 in FIG. 6. Here, it is assumed that a failure channel has been found in the transducer 110 by the self-diagnosis processing.

In step S802, the output part 183 of the ultrasonic probe 100 outputs information indicating that the transducer 110 has a failure using the output device 182.

In step S803, when the terminal 30 receives the operation to display the inspection result by the user, the ultrasonic diagnostic system 700 performs the processing from step S804. The user may perform the operation to display the inspection result only when the user recognizes that the ultrasonic probe 100 has a failure channel and requires further detailed information.

In step S804, the terminal 30 establishes wireless communication with the ultrasonic probe 100 using the communicator 180. In step S805, the terminal 30 transmits a request to obtain the inspection result to the ultrasonic probe 100 by wireless communication.

In step S806, the self-diagnosis circuit 210 of the ultrasonic probe 100 reads the inspection result from the storage 152. In step S807, the self-diagnosis circuit 210 transmits the read inspection result to the terminal 30 by wireless communication.

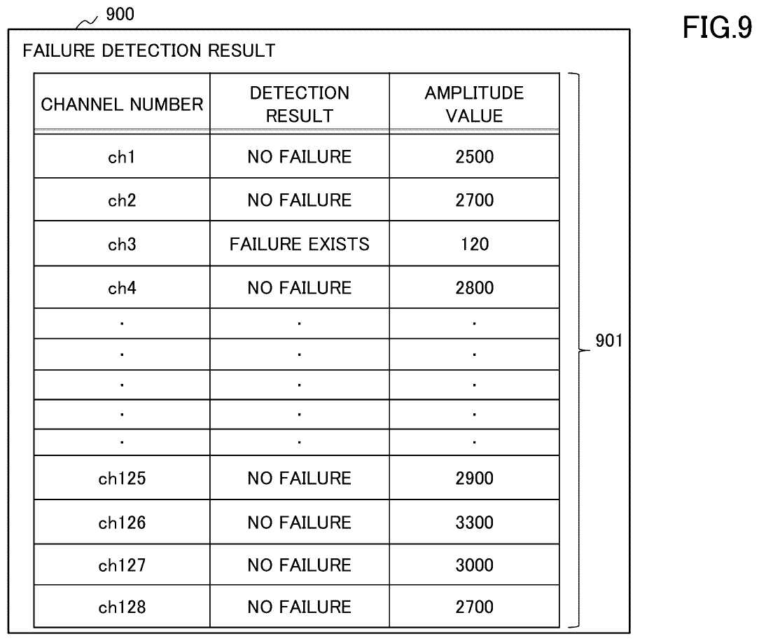

In step S808, the terminal 30 displays the inspection result received from the ultrasonic probe 100 on the display 162. For example, the terminal 30 displays, on the display 162, a display screen 900 displaying the inspection result 901 as illustrated in FIG. 9.

In the example of FIG. 9, the inspection result 901 includes, as items, a channel number, a detection result, and an amplitude value. The channel number is the channel number (ch1 to chN) of the transducer 110. The detection result is information indicating whether or not a failure has been detected in each channel. The amplitude value is, for example, the amplitude value of each channel calculated in step S306 of FIG. 6.

According to the second embodiment, a medical practitioner or the like who uses the ultrasonic probe 100 to diagnose the living body 2 can easily grasp the presence or absence of a failure of the ultrasonic probe 100 by simply pressing the switch for self-diagnosis of the ultrasonic probe 100.

A manager or the like who manages the ultrasonic probe 100 can easily view the detailed failure status of the ultrasonic probe 100 by using the terminal 30.

According to the embodiments of the present disclosure, the condition of the transducer can be diagnosed by the ultrasonic probe alone without depending on the display of the ultrasonic diagnostic system.

Thus, for example, when a plurality of ultrasonic probes 100 are in the hands of a medical practitioner or the like who is executing the diagnosis of the living body 2, the medical practitioner or the like can identify the non-defective ultrasonic probe 100 by simply pressing a switch for self-diagnosis without connecting the ultrasonic probe 100 to the terminal 30.

According to an embodiment of the present disclosure, the condition of the transducer can be diagnosed by the ultrasonic probe alone without using the display of the ultrasonic diagnostic system.

Although the embodiments of the present invention have been described above, the present invention is not limited to the specific embodiments, and various variations and changes can be made within the scope of the gist of the present invention as described in the claims.

What is claimed is:

1. An ultrasonic probe, comprising:
a transducer;
a self-diagnosis circuit configured to perform inspection of the transducer; and
an output circuit configured to output information that is in accordance with a result of the inspection performed by the self-diagnosis circuit.

2. The ultrasonic probe according to claim 1, further comprising:
a single housing, wherein
the transducer, the self-diagnosis circuit, and the output circuit are included in the single housing.

3. The ultrasonic probe according to claim 1, further comprising:
a storage configured to store the result of the inspection; and
a transmitter configured to transmit the result of the inspection to an exterior, the result of the inspection being read from the storage.

4. The ultrasonic probe according to claim 3, wherein
the transmitter is configured to transmit the result of the inspection to the exterior wirelessly.

5. The ultrasonic probe according to claim 3, wherein
the transmitter is configured to transmit the result of the inspection to the exterior by wired communication.

6. The ultrasonic probe according to claim 3, further comprising:
a single housing, wherein
the transducer, the self-diagnosis circuit, the output circuit, the storage, and the transmitter are included in the single housing.

7. The ultrasonic probe according to claim 1, wherein
the self-diagnosis circuit is configured to
sequentially cause a plurality of channels of the transducer to transmit and receive a signal one channel by one channel, and
determine that one or more of the channels have a failure in accordance with a difference between a maximum value and a minimum value of the signal received by the one or more of the channels within a predetermined period being smaller than a threshold value.

8. The ultrasonic probe according to claim 7, wherein
the output circuit is configured to output display information or sound information indicating whether or not the one or more of the channels of the transducer have a failure in accordance with the result of the inspection performed by the self-diagnosis circuit.

9. An ultrasonic diagnostic system, comprising:
the ultrasonic probe according to claim 3; and
a terminal, the terminal including a display and being configured to display, on the display, the result of the inspection received from the ultrasonic probe.

10. An ultrasonic diagnostic system, comprising:
the ultrasonic probe according to claim 4; and
a terminal, the terminal including a display and being configured to display, on the display, the result of the inspection received from the ultrasonic probe.

11. An ultrasonic diagnostic system, comprising:
the ultrasonic probe according to claim 5; and
a terminal, the terminal including a display and being configured to display, on the display, the result of the inspection received from the ultrasonic probe.

12. An ultrasonic diagnostic system, comprising:
the ultrasonic probe according to claim 6; and
a terminal, the terminal including a display and being configured to display, on the display, the result of the inspection received from the ultrasonic probe.

13. The ultrasonic diagnostic system according to claim 9, wherein
the result of the inspection includes
among a plurality of channels of the transducer, information on one or more of the channels that are determined to have a failure.

14. The ultrasonic diagnostic system according to claim 10, wherein
the result of the inspection includes
among a plurality of channels of the transducer, information on one or more of the channels that are determined to have a failure.

15. The ultrasonic diagnostic system according to claim 11, wherein
the result of the inspection includes
among a plurality of channels of the transducer, information on one or more of the channels that are determined to have a failure.

16. The ultrasonic diagnostic system according to claim 12, wherein
the result of the inspection includes
among a plurality of channels of the transducer, information on one or more of the channels that are determined to have a failure.
